(19) United States
(12) Patent Application Publication
(10) Pub. No.: US 2004/0115280 A1
(43) Pub. Date: Jun. 17, 2004

(54) Microcapsules

(76) Inventors: Wolfgang Podszun, Koln (DE); Joachim Kruger, Monheim (DE); Joachim Probst, Koln (DE)

Correspondence Address: Bayer Cropscience, Patent Department, 100 Bayer Road, Pittsburgh, PA, 15205-9741, US

(21) Appl. No.: 10/474,123

(22) PCT Filed: Apr. 2, 2002
(86) PCT No.: PCT/EP02/03617

(30) Foreign Application Priority Data
Apr. 10, 2001 (DE) 101 17 784.4

(51) Int. Cl. A01N 25/28; A61K 9/16; A61K 9/50; B01J 13/04

(52) U.S. Cl. 424/490; 504/359; 264/4.1

(57) ABSTRACT

Novel microcapsules comprising

a coat of polyurethane and/or polyurea and

a core of at least one solid active substance,

where the core does not comprise any liquid,

a process for preparing these novel microcapsules and their use for applying the active substances contained therein.

The present invention relates to novel microcapsules comprising as core solid active substances, to a process for preparing these microcapsules and for their use for applying the active substances contained therein.

Microcapsules are to be understood as meaning particles having a particle size of from about 1 to 200 μm and a core/coat structure, where the core is an active substance or comprises an active substance. Suitable active substances are, for example, pharmaceutically active compounds, agrochemically active compounds, flavors, additives, adhesives, dyes, leuco dyestuffs and flameproofing agents. The material of the coat can be a natural polymer, such as, for example, gelatin or gum arabic, or a synthetic polymer. Details about microencapsulation are described in Kirk-Othmer, “Encyclopedia of Chemical Technology”, Fourth Edition, Volume 16, pages 628-651.

Particularly suitable materials for the coat of microcapsules are polyurethanes and polyureas. Thus, microcapsules have been disclosed whose coat preferably consists of polyurea and whose core is filled with a suspension of solid biologically active compounds in a non-aqueous liquid (cf. WO 95-13 698). On the one hand, in the case of these microcapsules, the presence of a non-aqueous liquid in the core is required because otherwise formation of the coat by phase interface reaction would not be possible. On the other hand, the presence of non-aqueous liquids in the microcapsules is unfavorable with a view to their use, for the following reasons:

The content of active compounds in the microcapsules is reduced by the proportion of liquid.

On use, liquid may cause an unwanted effect, for example in the case of agro-chemical applications a contamination of the treated areas with the liquids.

The mechanical stability of the microcapsules is reduced by the liquid.

Novel microcapsules comprising

a coat of polyurethane and/or polyrea and

a core of at least one solid active substance

have now been found, where the core does not comprise any liquid.

Furthermore, it has been found that microcapsules according to the invention can be prepared by bringing a suspension of at least one solid active substance in water into contact with

a) at least one polyisocyanate dispersed in water and

b) at least one polyol and/or polyamine component.

Finally, it has been found that the microcapsules according to the invention are highly suitable for applying the solid active substances contained therein in the applications in question.

It is extremely surprising that the microcapsules according to the invention are more suitable for applying the solids contained therein than the constitutionally most similar preparations of the prior art. It is particularly unexpected that the microcapsules according to the invention, which consist virtually only of solids, release the core materials in the manner desired in each case.

The microcapsules according to the invention have a number of advantages. Thus, they comprise a very high proportion of active substances and are mechanically stable. Moreover, when using these microcapsules in agriculture, for example, there is no risk of a contamination of the treated areas with unwanted liquids.

As already mentioned, the coats of the microcapsules according to the invention consist of polyurethane and/or polyurea. These materials of the coat are derived from water-dispersible polyisocyanates which react with polyol and/or polyamine components. Monomers and polymers suitable for producing these materials of the coat are mentioned in connection with the description of the process according to the invention.

Suitable solid active substances which are contained in the microcapsules according to the invention as core materials are pharmaceutically active substances, agro-chemically active substances, flavors, additives, adhesives, leuco dyestuffs and flameproofing agents which are in each case solid at room temperature.

In the present context, agrochemical substances are to be understood as meaning all substances which are customary for crop treatment and whose melting point is above 20° C. Fungicides, bactericides, insecticides, acaricides, nematicides, molluscicides, herbicides, plant growth regulators, plant nutrients and repellents may be mentioned as being preferred.

Examples of fungicides which may be mentioned are:

2-anilino-4-methyl-6-cyclopropylpyrimidine; 2′,6′-dibromo-2-methyl-4′-trifluoromethoxy-4′-trifluoromethyl-1,3-thiazole-5-carboxanilide; 2,6-dichloro-N-(4-trifluoro-methylbenzyl)benzamide; (E)-2-methoximino-N-methyl-2-(2-phenoxyphenyl)-acetamide; 8-hydroxyquinoline sulfate; methyl (E)-2-{2-[6-(2-cyanophenoxy)-pyrimidin4-yloxy]phenyl}-3-methoxyacrylate; methyl (E)-methoximino[alpha-(o-tolyloxy)-o-tolyl]acetate; 2-phenylphenol (OPP), aldimorph, ampropylfos, anilazine, azaconazole,

benalaxyl, benodanil, benomyl, binapacryl, biphenyl, bitertanol, blasticidin-S, bromuconazole, bupirimate, buthiobate,

calcium polysulfide, captafol, captan, carbendazim, carboxin, quinomethionate, chloroneb, chloropicrin, chlorothalonil, chlozolinate, cufraneb, cymoxanil, cyproconazole, cyprofuram, carpropamid,

dichlorophen, diclobutrazol, diclofluanid, diclomezin, dicloran, diethofencarb, difenoconazole, dimethirimol, dimethomorph, diniconazole, dinocap, diphenylamine, dipyrithion, ditalimfos, dithianon, dodine, drazoxolon,

edifenphos, epoxyconazole, ethirimol, etridiazole,

fenarimol, fenbuconazole, fenfuram, fenitropan, fenpiclonil, fentin acetate, fentin hydroxide, ferbam, ferimzone, fluazinam, fludioxonil, fluoromide, fluquinconazole, flusilazole, flusulfamide, flutolanil, flutriafol, folpet, fosetyl-aluminum, fthalide, fuberidazole, furalaxyl, furmecyclox, fenhexamid,

guazatine,

hexachlorobenzene, hexaconazole, hymexazol,

imazalil, imibenconazole, iminoctadine, iprobenfos (IBP), iprodione, isoprothiolane, iprovalicarb,

kasugamycin, copper preparations, such as: copper hydroxide, copper naphthenate, copper oxychloride, copper sulfate, copper oxide, oxine copper and Bordeaux mixture,

mancopper, mancozeb, maneb, mepanipyrim, mepronil, metalaxyl, metconazole, methasulfocarb, methfuroxam, metiram, metsulfovax, myclobutanil,

nickel dimethyldithiocarbamate, nitrothal-isopropyl, nuarimol,

ofurace, oxadixyl, oxamocarb, oxycarboxin,

pefurazoate, penconazole; pencycuron, phosdiphen, pimaricin, piperalin, polyoxin, probenazole, prochloraz, procymidone, propamocarb, propiconazole, propineb, pyrazophos, pyrifenox, pyrimethanil, pyroquilon,

quintozene (PCNB), quinoxyfen,

sulfur and sulfur preparations,

tebuconazole, tecloftalam, techazene, tetraconazole, thiabendazole, thicyofen, thiophanate-methyl, thiram, tolclophos-methyl, tolylfluanid, triadimefon, triadimenol, triazoxide, trichlamide, tricyclazole, tridemorph, triflumizole, triforine, triticonazole, trifloxystrobin,

validamycin A, vinclozolin,

zineb, ziram,

2-[2-(1-chlorocyclopropyl)-3-(2-chlorophenyl)-2-hydroxypropyl]-2,4-dihydro-[1,2,4]-triazol-3-thione and

1-(3,5-dimethylisoxazole-4-sulfonyl)-2-chloro-6,6-difluoro-[1,3]-dioxolo-[4,5-f]-benzimidazole.

Examples of bactericides which may be mentioned are:

bronopol, dichlorophen, nitrapyrin, nickel dimethyldithiocarbamate, kasugamycin, octhilinone, furanecarboxylic acid, oxytetracyclin, probenazole, streptomycin, tecloftalam, copper sulfate and other copper preparations.

Examples of insecticides, acaricides and nematicides which may be mentioned are:

abamectin, acephate, acrinathrin, alanycarb, aldicarb, alphamethrin, amitraz, avermectin, AZ 60541, azadirachtin, azinphos A, azinphos M, azocyclotin,

Bacillus thuringiensis, 4-bromo-2-(4-chlorophenyl)-1-(ethoxymethyl)-5-(trifluoromethyl)-1H-pyrrole-3-carbonitrile, bendiocarb, benfuracarb, bensultap, betacyfluthrin, bifenthrin, BPMC, brofenprox, bromophos A, bufencarb, buprofezin, buto-carboxin, butylpyridaben,

cadusafos, carbaryl, carbofuran, carbophenothion, carbosulfan, cartap, chloethocarb, chloretoxyfos, chlorfenvinphos, chlorfluazuron, chlormephos, N-[(6-chloro-3-pyridinyl)methyl]-N′-cyano-N-methylethanimidamide, chlorpyrifos, chlorpyrifos M, cis-resmethrin, clocythrin, clofentezine, cyanophos, cycloprothrin, cyfluthrin, cyhalothrin, cyhexatin, cypermethrin, cyromazine,

deltamethrin, demeton-M, demeton-S, demeton-S-methyl, diafenthiuron, diazinon, dichlofenthion, dichlorvos, dicliphos, dicrotophos, diethion, diflubenzuron, dimethoate, dimethylvinphos, dioxathion, disulfoton,

emamectin, esfenvalerate, ethiofencarb, ethion, ethofenprox, ethoprophos, etrimphos,

fenamiphos, fenazaquin, fenbutatin oxide, fenitrothion, fenobucarb, fenothiocarb, fenoxycarb, fenpropathrin, fenpyrad, fenpyroximate, fenthion, fenvalerate, fipronil, fluazuron, flucycloxuron, flucythrinate, flufenoxuron, flufenprox, fluvalinate, fonophos, formothion, fosthiazate, fubfenprox, furathiocarb,

HCH, heptenophos, hexaflumuron, hexythiazox,

imidacloprid, iprobenfos, isazophos, isofenphos, isoprocarb, isoxathion, ivermectin,

lambda-cyhalothrin, lufenuron,

malathion, mecarbam, mevinphos, mesulfenphos, metaldehyde, methacrifos, methamidophos, methidathion, methiocarb, methomyl, metolcarb, milbemectin, monocrotophos, moxidectin,

naled, NC 184, nitenpyram,

omethoate, oxamyl, oxydemethon M, oxydeprofos,

parathion A, parathion M, permethrin, phenthoate, phorate, phosalone, phosmet, phosphamidon, phoxim, pirimicarb, pirimiphos M, pirimiphos A, profenofos, promecarb, propaphos, propoxur, prothiofos, prothoate, pymetrozin, pyrachlophos, pyridaphenthion, pyresmethrin, pyrethrum, pyridaben, pyrimidifen, pyriproxifen, quinalphos,

salithion, sebufos, silafluofen, sulfotep, sulprofos,

tebufenozide, tebufenpyrad, tebupirimiphos, teflubenzuron, tefluthrin, temephos, terbam, terbufos, tetrachlorvinphos, thiacloprid, thiafenox, thiamethoxam, thiodicarb, thiofanox, thiomethon, thionazin, thuringiensin, tralomethrin, transfluthrin, triarathen, triazophos, triazuron, trichlorfon, triflumuron, trimethacarb,

vamidothion, XMC, xylylcarb, zetamethrin.

Examples of molluscicides which may be mentioned are metaldehyde and methiocarb.

Examples of herbicides which may be mentioned are:

anilides such as, for example, diflufenican and propanil; arylcarboxylic acids such as, for example, dichloropicolinic acid, dicamba and picloram; aryloxyalkanoic acids such as, for example, 2,4-D, 2,4-DB, 2,4-DP, fluroxypyr, MCPA, MCPP and triclopyr; aryloxyphenoxyalkanoic esters such as, for example, diclofop-methyl, fenoxaprop-ethyl, fluazifop-butyl, haloxyfop-methyl and quizalofop-ethyl; azinones such as, for example, chloridazon and norflurazon; carbamates such as, for example, chlorpropham, desmedipham, phenmedipham and propham; chloroacetanilides such as, for example, alachlor, acetochlor, butachlor, metazachlor, metolachlor, pretilachlor and propachlor; dinitroanilines such as, for example, oryzalin, pendimethalin and trifluralin; diphenyl ethers such as, for example, acifluorfen, bifenox, fluoroglycofen, fomesafen, halosafen, lactofen and oxyfluorfen; ureas such as, for example, chlortoluron, diuron, fluometuron, isoproturon, linuron and methabenzthiazuron; hydroxylamines such as, for example, alloxydim, clethodim, cycloxydim, sethoxydim and tralkoxydim; imidazolinones such as, for example, imazethapyr, imazamethabenz, imazapyr and imazaquin; nitriles such as, for example, bromoxynil, dichlobenil and ioxynil; oxyacetamides such as, for example, mefenacet; sulfonylureas such as, for example, amidosulfuron, bensulfuron-methyl, chlorimuron-ethyl, chlorsulfuron, cinosulfuron, metsulfuron-methyl, nicosulfuron, primisulfuron, pyrazosulfuron-ethyl, thifensulfuron-methyl, triasulfuron and tribenuron-methyl; thiolcarbamates such as, for example, butylate, cycloate, diallate, EPTC, esprocarb, molinate, prosulfocarb, thiobencarb and triallate; triazines such as, for example, atrazine, cyanazine, simazine, simetryne, terbutryne and terbutylazine; triazinones such as, for example, hexazinone, metamitron and metribuzin; others such as, for example, aminotriazole, benfuresate, bentazone, cinmethylin, clomazone, clopyralid, difenzoquat, dithiopyr, ethofumesate, fluorochloridone, glufosinate, glyphosate, isoxaben, pyridate, quinchlorac, quinmerac, sulphosate and tridiphane. Mention may also be made of 4-amino-N-(1,1-dimethylethyl)-4,5-dihydro-3-(1-methylethyl)-5-oxo-1H- 1,2,4-triazole-1-carboxamide and 2-((((4,5-dihydro-4-methyl-5-oxo-3-propoxy-1H-1,2,4-triazol- 1-yl)carbonyl)amino)sulfonyl)methyl benzoate.

Plant growth regulators which may be mentioned are chlorocholine chloride and ethephon.

Examples of plant nutrients which may be mentioned are customary inorganic or organic fertilizers for providing plants with macro- and/or micronutrients.

Examples of repellents which may be mentioned are diethyltolylamide, ethylhexanediol and butopyronoxyl.

In the present context, flameproofing agents are to be understood as meaning substances having a melting point above 20° C. which can be incorporated into plastics and reduce their flammability. Examples which may be mentioned are halogen compounds which are solid at temperatures of up to 40° C. and the red form of phosphorus.

For preparing the microcapsules according to the invention, water-dispersible poly-isocyanates and polyol and/or polyamine components are required as starting materials for forming the materials of the coat.

In the present case, water-dispersible polyisocyanates are to be understood as meaning organic polyisocyanates which are liquid at room temperature and have free, aliphatically, cycloaliphatically and/or aromatically attached isocyanate groups.

Preference is given to polyisocyanates having a (mean) NCO functionality of from 2 to 5. Examples which may be mentioned are: m-phenylene diisocyanate, p-phenylene diisocyanate, toluylene 2,4-diisocyanate, 3,3′-dimethylbiphenylene 4,4′-diisocyanate, 4,4′-methylenebis(2-methylphenyl isocyanate), hexamethylene diisocyanate, trimethylhexamethylene diisocyanate, 4,4′-methylenebis(cyclohexyl isocyanate). Likewise highly suitable are derivatives of diisocyanates having biuret, urethane, uretdione and/or isocyanurate groups, for example the trimeric hexamethylene diisocyanate having isocyanurate structure which can be obtained according to U.S. Pat. No. 4 324 879.

Also suitable are polyisocyanates which have been rendered hydrophilic and which can be obtained from the polyisocyanates mentioned above by partial reaction of the NCO groups with ionic or nonionic compounds, for example by reaction with polyethylene oxide. Particularly suitable hydrophilic polyisocyanates are disclosed in EP-A 0 959 087. Such hydrophilic polyisocyanates have the advantage that they are self-dispersing. However, the process according to the invention is not limited to the use of these polyisocyanate types. Polyisocyanates which have not been rendered hydrophilic can be emulsified with the aid of polyol components, which are likewise required as starting materials, or with other surface-active agents.

Polyol components suitable for carrying out the process according to the invention are polymers having both hydroxyl groups and carboxylate and/or sulfonate groups. These include, for example, polymers of olefinically unsaturated compounds which contain hydroxyl groups.

Preference is given to hydroxyl-group-containing polymers which have a molecular weight Mₙ (number average), determined by gel permeation chromatography, of from 500 to 50 000, preferably from 1000 to 10 000, and a hydroxyl number of from 16.5 to 264, preferably from 33 to 165, mg of KOH/g of polymer. In addition to hydroxyl groups, the polyol component also comprises carboxylate and/or sulfonate groups, the proportion of these groups being from 5 to 500, preferably from 25 to 250, milliequivalent/100 g of polymer. The carboxylate and/or sulfonate groups increase the solubility in water or the dispersibility of the polymers.

The hydroxyl-group-containing polymers can be prepared by copolymerization using hydroxyl-group-containing monomers and monomers which contain carboxylic acid groups and/or sulfonic acid groups, at least some of the carboxylic acid groups and/or sulfonic acid groups being neutralized after the polymerization has been carried out. Preferred hydroxyl-group-containing monomers are, for example, 2-hydroxyethyl methacrylate, 2-hydroxyethyl acrylate, hydroxypropyl acrylate and hydroxypropyl methacrylate. Examples of monomers having carboxylic acid groups are acrylic acid, methacrylic acid, maleic acid and itaconic acid. A suitable monomer having a sulfonic acid group is 2-acrylamido-2-methylpropanesulfonic acid. Further monomers which can be used for preparing the hydroxyl-group-containing polymers are monomers without functional groups, such as, for example, methyl methacrylate, methyl acrylate, ethyl acrylate, ethyl methacrylate, isopropyl methacrylate, isopropyl acrylate, n-propyl acrylate, n-butyl methacrylate, n-butyl acrylate, 2-ethylhexyl acrylate, 2-ethylhexyl methacrylate, stearyl methacrylate, styrene, acrylonitrile, methacrylonitrile, vinyl acetate and vinyl propionate. The amounts of hydroxyl-group-containing monomers and monomers having carboxylic acid groups and/or sulfonic acid groups are generally chosen such that the characterizing numbers given above are reached. Further details on how to prepare hydroxyl-group-containing polymers which are suitable for use as starting materials in the practice of the process according to the invention are given in EP-A 0 358 979.

When carrying out the process according to the invention, it is also possible to use polyamines instead of the polyols or, preferably, in addition to the polyols. Suitable polyamines of this type are, preferably, diethylenetriamine or triethylenetetramine.

When carrying out the process according to the invention, a finely divided suspension of one or more active substances in water is used. In this context, finely divided means that the particles have a mean particle size of between 1 and 200 μm, preferably between 2 and 100 μm. These suspensions can be prepared by comminuting the solid active substances with the aid of customary mills, such as bead mills or ball mills, followed by suspension in water. Here, it is advantageous to suspend the solid active substance in water which already contains the polyisocyanate or the polyol and/or polyamine component. Particularly preferably, dispersing is carried out in the presence of the polyol component. The polyisocyanate is then added once the suspension is as finely divided as desired.

Microencapsulation according to the invention is carried out by stirring in customary mixers.

In the practice of the process according to the invention, the temperatures can be varied within a certain range. In general, the reaction in which the capsule coats are formed is carried out at room temperature. However, it is also possible to operate at temperatures between 20° C. and 100° C.

It is also possible to add a catalyst to the reaction mixture to accelerate capsule formation when carrying out the process according to the invention. Here, suitable catalysts are, for example, organic tin compounds, such as dibutyltin dilaurate, or else tertiary amines, such as triethylamine. The concentration of catalyst can be varied within a certain range. In general, the catalyst is employed in amounts of between 0.01 and 0.5% by weight, based on the polyisocyanate.

The practice of the reaction according to the invention generally takes a number of hours. Here, it is possible to monitor the progress of the reaction by IR-spectroscopic detection of the NCO content.

When carrying out the process according to the invention, the ratio of polyisocyanate to polyol and/or polyamine component can be varied within a certain range. In general, polyisocyanate and polyol and/or polyamine component are employed in amounts such that an NCO/OH (NH) equivalent ratio of between 0.5:1 and 3:1 results.

It is also possible to vary the total amount of polyisocyanate and polyol and/or polyamine component, based on the solid active substance, within a certain range. In general, polyisocyanate and polyol and/or polyamine component are employed in such a total amount that the weight ratio of the components employed for forming the capsule coats to active substance is between 1:0.001 and 1:1, preferably between 1:0.01 and 1:0.25.

Depending on the particle size of the active substances used, the particle size of the microcapsules according to the invention can be varied within a relatively wide range. Accordingly, the particle size of the microcapsules is generally between 1 and 200 μm, preferably between 2 and 100 μm. Microcapsules comprising agrochemical substances as active substances particularly preferably have a mean particle size between 2 and 30 μm.

When carrying out the process according to the invention, the microcapsules according to the invention are obtained as solid particles in aqueous suspension. If it is desired to remove the microcapsules, the capsules can be isolated, for example, by filtration or decanting and, if appropriate after washing, dried.

If the microcapsules according to the invention comprise agrochemically active compounds, they are highly suitable for applying these active compounds to plants and/or their habitat. Here, the microcapsules according to the invention can be used in practice as such either in solid form or as suspensions, if appropriate after prior dilution with water and if appropriate after addition of formulation auxiliaries. Application is carried out by customary methods, i.e., for example, by watering, spraying, atomizing or broadcasting.

The application rate of microcapsules according to the invention which comprise agrochemically active compounds can be varied within a relatively wide range. It depends on the respective agrochemically active compounds and their content in the microcapsules.

The microcapsules according to the invention which comprise agrochemically active compounds ensure that the active compounds are released in the amount desired in each case over a relatively long period of time.

Microcapsules according to the invention which comprise flameproofing agents are easier to incorporate into plastics than non-encapsulated flameproofing agents. Moreover, by incorporating flameproofing agents which are microencapsuled according to the invention into plastics, it is possible to substantially avoid an undesirable effect on the properties of the plastics, for example on a reduction of the mechanical strength.

The invention is illustrated by the examples below.

### PREPARATION EXAMPLES

### Example 1

### Preparation of a Hydrophilicized Polyisocyanate

At 100° C., 870 g (4.88 val) of an isocyanurate-group-containing polyisocyanate based on 1,6-diisocyanatohexane having an NCO content of 23.2%, a mean NCO functionality of 3.2 (according to GPC), a content of monomeric 1,6-diisocyanato-hexane of 0.2% and a viscosity of 1200 mPas (23° C.) are initially charged under dry nitrogen and with stirring, 130 g (0.26 val) of a methanol-initiated, monofunctional polyethylene oxide polyether of a mean molecular weight of 500, corresponding to an NCO/OH equivalent ratio of 18.5:1 are added over 30 min and the mixture is then stirred at this temperature until the NCO content of the mixture has, after about 2 h, decreased to a value of 19.1%, which corresponds to complete urethenization. The allophanatization reaction is started by addition of 0.01 g of zinc(II) 2-ethyl-1-hexanoate. Owing to the heat of reaction released, the temperature of the reaction mixture increases up to 109° C. After the exothermic reaction has subsided, about 20 min after the addition of the catalyst, the reaction is terminated by addition of 0.01 g of benzoyl chloride and the reaction mixture is cooled to room temperature. This gives a hydrophilicized polyisocyanate having a solids content of 100%. The isocyanate content is 18.1%, the functionality is 3.8, the NCO equivalent weight is 232 g and the viscosity at a shear rate of D=40 s⁻, at 4000 mPa·s.

### Example 2

### Preparation of a Polyol Component

By reacting the comonomers 2-hydroxyethyl methacrylate, acrylic acid, methyl methacrylate and 2-butyl acrylate by the process described in EP-B 0 358 979, a polyacrylate in the form of a secondary dispersion was prepared. The dispersion has a solids content of 46%, an OH content of 3.3% with respect to solid resin, an acid number of about 21 mg KOH/g of solid resin, a pH of 8.0 and a viscosity of about 800 mPa·s (23° C., D=40 s⁻¹). The neutralizing agent used is N-dimethylamino-ethanol.

### Example 3

### Microencapsulation of Red Phosphorus

40 g of red phosphorus having a mean particle size of 35 μm were suspended in 337 g of deionized water which contained 17.4 g of the polyol component described in example 2. With stirring at room temperature, 5.4 g of the polyisocyanate mentioned in example 1 and 8 mg of dibutyltin bis(1-thioglycerol)=(Fascat 4224; from: Elf Atochem Inc., Industrial Specialties) as catalyst were then added. The stirrer speed was adjusted to 300 rpm and the reaction mixture was then heated at 50° C. for 18 hours. After cooling of the reaction mixture to room temperature, the microencapsulated product was isolated by filtration, washed with 250 ml of water and then dried at 60° C. until the weight remained constant. This gave 29.9 g of microencapsulated product which did not smell of phosphine.

### Example 4

### Microencapsulation of Red Phosphorus

The reaction described in example 3 was repeated without addition of catalyst. The reaction time at 50° C. was 42 hours. After drying, 30.8 g of microencapsulated product, which did not smell of phosphine, were obtained.

### Example 5

### Microencapsulation of Red Phosphorus

40 g of red phosphorus of a mean particle size of 35 μm were suspended in 350 g of deionized water. 8.72 g of the polyisocyanate described in example 1 were then added. The stirrer speed was adjusted to 300 rpm and 1.29 g of diethylenetriamine were then added dropwise. Subsequently, the reaction mixture was, with stirring, heated at 50° C. for 18 hours. After cooling of the reaction mixture to room temperature, the microencapsulated product was isolated by filtration, washed twice with 200 ml of water and then dried at 60° C. until the weight remained constant. This gave 44.1 g of microencapsulated product which did not smell of phosphine.

### Example 6

### Microencapsulation of Thiacloprid

A suspension of 40 g of thiacloprid in 337 g of deionized water was treated successively with 17.4 g of the polyol component described in example 2, 5.4 g of the polyisocyanate described in example 1 and 8 mg of the catalyst mentioned in example 3. The stirrer speed was adjusted to 300 rpm and the reaction mixture was then stirred at room temperature for 18 hours. The microencapsulated product was subsequently isolated by centrifugation, washed thoroughly with water, centrifuged again and then dried at 60° C. until the weight remained constant. This gave 35.5 g of pulverulent microencapsulated product.

### Example 7

### Microencapsulation of Imidacloprid

40 g of imidacloprid were microencapsulated under the conditions stated in example 6. This gave 37.6 g of pulverulent microencapsulated product.

### Example 8

### Microencapsulation of Thiacloprid

20 g of imidacloprid were suspended in a mixture of 169 g of deionized water and 17.4 g of the polyol component described in example 2. 5.4 g of the polyisocyanate described in example 1 and 8 mg of the catalyst mentioned in example 3 were added to the resulting suspension. The stirrer speed was adjusted to 300 rpm and the reaction mixture was then stirred at room temperature for 24 hours. The microencapsulated product was then isolated by centrifugation, washed thoroughly with water, centrifuged again and then dried at 60° C. until the weight remained constant. This gave 19.8 g of pulverulent microencapsulated product.

### Use Example A

### Stability Test

To test the thermal stability of microcapsules according to the invention, samples of in each case 100 mg of the products described in examples 3 to 5 were heated in a closed apparatus at 250° C. for 10 minutes. The amount of phosphine formed was determined. For comparison, starting material (red phosphorus) which had not been encapsulated was examined, too.

The test results are shown in the table below.

TABLE A

| Product | | |
|---|---|---|
| according to | | Phosphine release |
| example | Smell of phosphine | after 10 min at 250° C. |
| red phosphorus | strong | 400 ppm |
| (comparison) | | |

According to the invention:

| | | |
|---|---|---|
| (3) | — | 22 ppm |
| (4) | — | 25 ppm |
| (5) | — | 28 ppm |

1. A microcapsule, comprising
a coat of polyurethane and/or polyurea and
a core of at least one solid active substance,
where the core does not comprise any liquid.

2. A microcapsule as claimed in claim 1, characterized in that it comprises, as active substances, pharmaceutically active compounds, agrochemically active compounds, flavors, additives, adhesives, leuco dyestuffs or flameproofing agents having a melting point above 20° C.

3. A microcapsule as claimed in claim 1, characterized in that it comprises, as active substances, fungicides, bactericides, insecticides, acaricides, nematicides, molluscicides, herbicides, plant growth regulators, plant nutrients and/or repellents having a melting point above 20° C.

4. A microcapsule as claimed in claim 1, characterized in that it comprises, as active substance, imidacloprid.

5. A microcapsule as claimed in claim 1, characterized in that it comprises, as active substance, thiacloprid.

6. A process for preparing microcapsules as claimed in claim 1, characterized in that a suspension of at least one solid active substance in water is brought into contact with
a) at least one polyisocyanate dispersed in water and
b) at least one polyol and/or polyamine component.

7. The use of microcapsules as claimed in claim 1 for applying the solid active substances contained therein.
